(19) United States
(12) Patent Application Publication
(10) Pub. No.: US 2022/0134027 A1
(43) Pub. Date: May 5, 2022

(54) Dose Feedback Mechanisms and Assemblies for User Feedback

(71) Applicant: Lupin Inc., Coral Springs, FL (US)

(72) Inventors: Xian-Ming Zeng, Coral Springs, FL (US); Imran Shaikh, Coral Springs, FL (US); Abhishek Gupta, Weston, FL (US)

(21) Appl. No.: 17/429,030

(22) PCT Filed: Feb. 5, 2020
(86) PCT No.: PCT/US2020/016784

§ 371 Date: Aug. 6, 2021

Related U.S. Application Data
(60) Provisional application No. 62/801,697, filed on Feb. 6, 2019.

(51) Int. Cl. A61M 15/00; G06M 1/04

(52) U.S. Cl. CPC A61M 15/0075; G06M 1/045; A61M 15/0081

(57) ABSTRACT

The present disclosure relates to a medicament dispenser for dispensing medicament. The dispenser may be for use in dispensing medicament in a liquid, pressurized aerosol, pre-filled container and in any medicament dispenser where dose related feedback is required. Mechanisms and assemblies provide dose related feedback to the user of the medicament dispenser and the physical interfaces and attributes associated with providing such feedback. The present disclosure also relates to dose feedback assemblies that can be adapted to various form factors, various individual components and elements of the container-closure system, and/or various target user interfaces. The dispenser is suited for dispensing propellant based pressurized inhalation aerosols for oral and/or nasal delivery; aqueous or non-aqueous systems for oral and/or nasal delivery; liquid dispensers for nasal delivery, multi-dose pre-filled syringes or pens; or dispensers for intra-muscular or subcutaneous delivery and powders for pulmonary administration and tablets, capsules, pellets or agglomerates for oral administration. The dispenser may dispense a propellant based pressurized inhalation aerosol comprising one or more active pharmaceutical ingredient(s) (API) and optionally, one or more propellant, cosolvent, solubilizer, emulsifier, surfactant, salt, acid and micronized or non-micronized pharmaceutically acceptable carrier(s) and/or excipient(s), wherein the dispensing process includes a press and breathe type or breath actuated or other multi-step operation medicament dispensers.

### FIELD

The present disclosure relates to a medicament dispenser for dispensing medicament. Mechanisms and assemblies provide dose related feedback to the user of the medicament dispenser and the physical interfaces and attributes associated with providing such feedback.

### BACKGROUND

Dose indicator or counters are useful in a wide variety of applications, and are especially important in the field of medicament dispensers where an accurate determination of the number of doses of medicament remaining in a medicament container might otherwise be difficult to obtain. Examples of such a medical dispenser includes liquid, pressurized aerosol, pre-filled containers and in any medicament dispenser where dose related feedback is required, e.g., pressurized metered-dose inhaler (pMDI), dry-powder inhaler (DPI), pre-filled container, tablet/capsule dispenser, etc.

Metered dose inhalers (MDI) are devices that delivers a specific amount of medication to the lungs, in the form of a short burst of aerosolized medicine that is usually self-administered by the patient via inhalation. Metered-dose inhalers include pressurized metered-dose inhalers and dry-powder inhalers. Generally, pMDIs include three major components; an aerosol canister where the formulation resides for administration to the lungs, a metering valve which is disposed in the canister and which allows a metered quantity of the formulation to be dispensed with each actuation, and an actuator which holds the canister and allows the patient to operate the device and directs the aerosol into the patient's lungs. Dry powder inhalers are devices that delivers a specific amount of medication to the lungs, in the form of a dry powder.

Metered dose inhalers are used in order to administer an accurate dose of medicament. A more recent development is the so-called “breath actuated inhaler” which delivers a dose of drug through a mouthpiece in response to inhalation by the user. BDIs are preferred in circumstances where the co-ordination between user inhalation and manual depression of the aerosol canister is imperfect. For example, children have difficulty synchronizing actuation of the MDI with inhalation. Sometimes patient breathes out before inhalation is complete.

Unfortunately, one of the drawbacks of self-administration from conventional inhaler is that they provide no convenient way for patients to track the number of doses remaining in the canister at any given time. Thus, the illusion is created that the inhaler is still capable of providing useful doses of medicament simply because the canister contains liquid. This is potentially hazardous for the user since dosing becomes unreliable.

Thus, integration of dose-counting mechanisms into MDI drug products enables users to assess how many doses remain in the obscured canister. It is recommended that manufacturers integrate a dose-counting device into new MDIs as either a numerical countdown indicating the number of remaining doses or as color-coding indicating the device should not be used.

U.S. Pat. No. 5,349,945 includes a counting device for aerosol dispensers with a rotatable display means having a rack of teeth which is driven by a ratchet during the dispensing of a medicament dose. Each tooth on the rack corresponds to a single dose. However, miscounting might occur with one poor tooth. This requires all of the teeth in the rack to be perfect.

U.S. Pat. No. 5,988,496 describes a device for counting doses of substance issued by a dispenser. The device includes a first count wheel and second count wheel mounted to rotate about a common axis of rotation. The first count wheel includes a drive tongue that is movable between a rest position, in which it does not cooperate with the second count wheel, and a drive position, in which it cooperates with the second count wheel to cause it to rotate about the common axis of rotation. The drive tongue is forced into position by action of a cam. Such device might possess difficulty in robust transfer and distribution of motion upon actuation.

Although such devices have provided the advantage of being able to provide some measure of the number of doses of medicament dispensed from a container and/or the number of doses remaining therein, there remains room for improvement. In particular, it has proven difficult to provide dose counters that reliably “count” the release of medicament doses from containers. Moreover there is also regulatory pressure to minimize the number of false counts. Naturally there is a need to develop a dose feedback mechanism that is efficient and robust.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavor to which this specification relates.

### SUMMARY

The present disclosure provides for mechanisms and assemblies for providing dose related feedback to the user of the medicament dispenser and the physical interfaces and attributes associated with providing such feedback. The dispenser may dispense medicament in a liquid, pressurized aerosol, pre-filled container, or in any medicament dispenser where dose related feedback is required. The present disclosure also relates dose feedback assemblies that can be adapted to various form factors, various individual components and elements of the container-closure system, and/or various target user interfaces. The dispenser is suited for dispensing propellant based pressurized inhalation aerosols for oral and/or nasal delivery; aqueous or non-aqueous systems for oral and/or nasal delivery; liquid dispensers for nasal delivery and powders for pulmonary administration and tablets, capsules, pellets or agglomerates for oral administration; or pre-filled syringes or pens or dispensers for intra-muscular or subcutaneous delivery. The dispenser may dispense a propellant-based pressurized inhalation aerosol comprising one or more active pharmaceutical ingredient(s) (API) and optionally, one or more propellant, cosolvent, solubilizer, emulsifier, surfactant, salt, acid and micronized or non-micronized pharmaceutically acceptable carrier(s) and/or excipient(s), wherein the dispensing process includes a press and breathe-type or breath-actuated or other multi-step operation medicament dispensers. The present disclosure also relates to dry powder inhaler-type devices comprising one or more active pharmaceutical ingredient(s) and optionally, one or more micronized or non-micronized pharmaceutically acceptable carrier(s) and/or excipient(s) wherein the dispensing process includes multi-step operation medicament dispensers such as open, dispense, and close.

In some embodiments, the mechanism of actuation leads to change in the number of doses (increment or decrement) and provides dose related feedback (e.g., number remaining, number consumed, and/or life-cycle of the product) to the user of the medicament dispenser.

In some embodiments, the mechanism of actuation leads to change in the number of doses (increment or decrement) and also leads to the exhaustion/evacuation of the contents of the primary packaging to provide dose-related feedback (e.g., number remaining, number consumed, and/or life-cycle of the product) to the user of the medicament dispenser.

In some embodiments, the dose count feedback is provided through a dose indicator assembly, wherein dose feedback mechanism includes a mechanism by which the dose-related (e.g., number remaining, number consumed, and/or life-cycle of the product) visual feedback (e.g., readout) is provided to the use.

In some embodiments, the dose feedback mechanism is provided through a dose indicator assembly.

In some embodiments, the dose feedback mechanism is provided through a dose indicator assembly which further comprises dose indicating means.

In some embodiments, the dose feedback mechanism is provided through a dose indicator assembly including wheel(s) or disk(s).

In some embodiments, a ‘window’ (or interface or display) provides the dose-related (e.g., number remaining, number consumed, and/or life-cycle of the product) visual feedback (e.g., readout) to the user of the medicament dispenser.

In some embodiments, the dose count feedback ranges from numbers ‘0’ to ‘999’ which are displayed synchronously, collectively and simultaneously with single or multiple components of the dose indicator means through the window (or interface or display).

In some embodiments, a large window (or interface or display) displays the individual digits (e.g., numerical feedback) in a vertical or horizontal orientation and provides for the dose related (e.g., number remaining, number consumed, and/or life-cycle of the product) visual feedback (e.g., read-out) to the user of the medicament dispenser.

In some embodiments, the dose feedback mechanism includes a dose indicator assembly having one or more dose indicator wheels.

In some embodiments, the dose feedback mechanism includes simultaneous and collective read-out of the digits (e.g. numerical feedback) imprinted or etched or pasted on one to three dose indicator wheels of dose indicating means through the window (or interface or display).

In some embodiments, the dose feedback mechanism includes at least two dose indicator wheels in a concentric, planar, and co-axial (e.g., same axis of rotation) orientation; a non-concentric and non-planar orientation; a concentric, co-planar, and co-axial orientation; overlapping orientation; or non-overlapping orientation. The dose feedback mechanism provides a simultaneous and collective read-out of the digits (e.g., numerical feedback) imprinted or etched or pasted or embossed on one or more dose-indicator wheels of dose-indicating means through the window (or interface or display).

In some embodiments, the dose feedback mechanism comprises of two to three dose indicator wheels of dose indicating means in a concentric, co-planar, overlapping and/or non-overlapping orientation. The dose feedback mechanism provides a simultaneous and collective read-out of the digits (e.g. numerical feedback) imprinted or etched or pasted on one or more dose indicator wheels of dose indicating means through the window (or interface or display).

In some embodiments, the dose feedback mechanism includes one or more dose indicator wheels that move synchronously and simultaneously with the movement of the primary packaging and provides collective read-out of the digits (e.g., numerical feedback) that are imprinted or etched or pasted or embossed on the one or more dose indicator wheels through the window (or interface or display).

In some embodiments, the dose feedback mechanism includes one or more dose indicator wheels that move synchronously and simultaneously with the movement of the primary packaging and with the aid of one or more stationary and rotating wheels or shafts and provides collective read-out of the digits (e.g., numerical feedback) that are imprinted or etched or pasted or embossed on the one or more substrates through the window (or interface or display).

In some embodiments, the dose feedback mechanism includes two or more dose indicator wheels in a concentric and co-planar orientation with a simultaneous and collective read-out of the digits (e.g. numerical feedback) imprinted or etched or pasted on one to three dose indicator wheels through the window (or interface or display).

In some embodiments, the dose feedback mechanism includes two or more dose indicator wheels that are in a concentric, coaxial (e.g. same axis of rotation) orientation; planar or non-planar orientation with respect to each other; connected directly or indirectly to each other; and/or with a simultaneous and collective read-out of the digits (e.g. numerical feedback) imprinted or etched or pasted on the one to three dose indicator wheels through the window (or interface or display).

In some embodiments, the dose feedback mechanism includes two or more dose indicator wheels that are in a concentric, coaxial (e.g., same axis of rotation) orientation, or in a planar or non-planar orientation with respect to each other. The two or more dose indicator wheels may be connected directly or indirectly to each other. All dose indicator wheels may move in the same direction; wherein at least two wheels move in the opposite direction with respect to each other. A simultaneous and collective read-out of the digits (e.g., numerical feedback) imprinted, etched, pasted, or embossed on the one or more dose indicator wheels is visible through the window (or interface or display).

In some embodiments, the dose feedback mechanism includes two or more dose indicator wheels that are in a concentric, coaxial (e.g. same axis of rotation) orientation, in a planar orientation with respect to each other. For example, the units wheel may be centrally located with the tens wheel positioned radially outward of the units wheel and the hundred wheel positioned radially outward of the tens wheel. In this configuration, the front faces of each dose indicator wheel are aligned along the same plane such that, when aligned, the digits can be viewed through a window on the actuator. A single digit from each dose indicator wheel can be viewed through the window in a vertical orientation. The two or more dose indicator wheels may be connected directly or indirectly to each other. All dose indicator wheels may move in the same direction; wherein at least two wheels move in the opposite direction with respect to each other. A simultaneous and collective read-out of the digits (e.g. numerical feedback) imprinted, etched, pasted, or embossed on the one or more dose indicator wheels is visible through the window (or interface or display).

In some embodiments, the dose feedback mechanism includes two or more dose indicator wheels moving synchronously, collectively, and simultaneously with the operation of the medicament dispenser and provides for the dose-related (e.g., number remaining, number consumed, and/or life-cycle of the product) visual feedback (e.g., read-out) to the user of the medicament dispenser.

In some embodiments, the dose feedback mechanism includes one or more dose indicator wheels with units digits, including tens and/or hundreds digits imprinted or etched or pasted or embossed on the dose indicator wheels.

In some embodiments, the dose feedback mechanism including one or more co-axial (e.g. same axis of rotation) wheels that move synchronously, collectively and simultaneously with the operation of the medicament dispenser.

In some embodiments, the dose feedback mechanism including one or more concentric (e.g. have a common center) wheels that move synchronously, collectively and simultaneously with the operation of the medicament dispenser.

In some embodiments, the dose feedback mechanism includes the synchronous, collective, and simultaneous readout of one or more wheels representing the units digits, the tens digits and/or the hundreds digits through the window (or interface or display) of the medicament dispenser.

In some embodiments, the dose feedback mechanism includes the operation of one or more linear rack(s) of dose indicator assembly transforming motion from actuation/operation of the medicament dispenser to the dose feedback mechanism of the medicament dispenser.

In some embodiments, the rectilinear motion of the primary packaging of the medicament dispenser during the actuation/operation of the medicament dispenser is converted to rotational motion of the dose feedback mechanism.

In some embodiments, the rectilinear motion of the mechanism for actuation/operation of the medicament dispenser is mechanically converted to rectilinear motion for the operation of the dose feedback mechanism and wherein this rectilinear motion is further converted to the rotational motion of the dose feedback mechanism of the medicament dispenser.

In some embodiments, the rotational motion of the mechanism for operating/opening/actuating the medicament dispenser is mechanically converted to rectilinear motion for the operation of the dose feedback mechanism and wherein this rectilinear motion is further converted to the rotational motion of the dose feedback mechanism of the medicament dispenser.

In some embodiments, the present invention provides for a medicament dispenser wherein the dose feedback mechanism is also directly connected to the mechanism of operating/opening/actuating the medicament dispenser.

In some embodiment, the dose feedback mechanism includes one or more linear racks having teeth; one or more pinions; a dose indicating means for providing a dose related visual feedback to the user of the medicament dispenser.

In some embodiment, the dose feedback mechanisms includes: one or more linear racks having teeth wherein rectilinear motion of the linear rack is converted in rotational motion through pinion and/or a dose indicating means for providing a dose related visual feedback to the user of the medicament dispenser wherein it comprises one or more dose indicator wheels arranged to provide a count ranging from numbers ‘0’ to ‘999’. The pinions rotates the dose indicator wheel to provide the synchronous, collective and simultaneous readout of the count.

In some embodiment, a dose indicator assembly includes a disc mount, one or more linear racks having teeth, one or more pinions and/or a dose indicating means for providing a dose related visual feedback to the user of the medicament dispenser.

In some embodiment, a dose indicator assembly includes a disc mount, one or more linear racks having teeth, one or more pinions and/or a dose indicating means including dose indicator wheels for providing a dose related visual feedback to the user of the medicament dispenser.

In some embodiments, the dose feedback mechanism includes one or more linear racks that allow for an efficient and robust transfer and distribution of motion from the actuation/operation of the medicament dispenser to and within the dose feedback mechanism. The dose feedback mechanism also allow for relative serial reduction in the unit count of doses remaining in the medicament dispenser.

In some embodiments, the dose feedback mechanism includes one or more linear racks that prevent an inadvertent miss in the transfer and distribution of motion of the primary packaging within the dose feedback mechanism and prevent a false higher remaining dose count within the medicament dispenser.

In some embodiments, the dose feedback mechanism includes one or more linear racks that prevent an inadvertent partial movement of one or more dose indicator wheels.

In some embodiments, the dose feedback mechanism provides features to prevent reverse movement of dose indication means in the medicament dispenser.

In some embodiments, the dose feedback mechanism provides features that lend its utility in a reusable dose feedback mechanism and in a separable cartridge or cassette for re-use and re-loading of the medicament dispenser.

In some embodiments, the dose indicator driving mechanism includes a circular readout of increments or decrements in the number of doses in the medicament dispenser and where the units, tens and hundreds digits are stacked vertically above each other for a collective, synchronous and simultaneous readout through the window (or interface or display) of the medicament dispenser.

In some embodiments, the dose indicator driving mechanism includes a large and intuitive dose indicator mechanism.

In some embodiments, the dose indicator driving mechanism includes dose indicating wheels in a plane which is at 180 degrees relative to the plane (i.e. frontal view) of the medicament dispenser.

In some embodiments, the dose feedback mechanism includes a mechanical dose indicating mechanism and an electronic dose indicating mechanism that operate in a synchronous, collective, tandem and redundant manner.

In some embodiments, the material of construction of the components of the dose feedback mechanism includes acrylonitrile butadiene styrene (ABS), polycarbonate/acrylonitrile butadiene styrene terpolymer blend (PC/ABS), Polyoxymethylene (POM), nylon, stainless steel and/or silicone rubber.

In some embodiments, the material of construction of the components of the dose feedback mechanism comprises of acrylonitrile butadiene styrene (ABS), polycarbonate/acrylonitrile butadiene styrene terpolymer blend (PC/ABS), Polyoxymethylene (POM) and nylon.

In some embodiments the dose indicator mechanism also provides for complete dose lock-out and complete operation lock-out features after all doses in the medicament dispenser are dispensed and the medicament dispenser is exhausted.

### BRIEF DESCRIPTION OF DRAWINGS

FIGS. 1A and 1B show an isometric view of embodiments of the medicament dispenser.

FIG. 2 shows a partial-exploded isometric view of a different embodiment of the medicament dispenser.

FIG. 3 shows an exploded view of a medicament dispenser showing an embodiment of a dose indicator assembly.

FIG. 4 shows the view of a window displaying the count of an embodiment of the dose indicator assembly.

FIG. 5A shows top isometric views of a medicament dispenser.

FIG. 5B shows a top isometric view of an actuator showing assembled bar type balancer.

FIG. 6 shows a sectional view showing the actuator, bar type balancer and the dose indicator assembly.

FIG. 7 shows an arrangement of linear rack inside the actuator.

FIG. 8 shows arrangements of dose indicator assembly and bar type balancer.

FIG. 9 shows an exploded view of the dose indicator assembly.

FIG. 10 shows an exploded view of an embodiment of the dose indicator assembly along with its operation.

FIG. 11 shows an illustration of dose lockout feature of the dose indicator assembly.

FIGS. 12A and 12B show different isometric views of a disc mount.

FIGS. 13A and 13B show different isometric views of a unit wheel.

FIGS. 14A and 14B show different isometric views of a tens wheel.

FIGS. 15A and 15B show different isometric views of a hundreds wheel.

FIG. 16 shows an isometric view of a central pinion.

FIG. 17 shows an isometric view of tens/hundreds wheel drive gear.

FIG. 18 shows an isometric view of a linear rack.

FIG. 19 shows an isometric views of a bar type balancer.

FIG. 20 shows an operating cycle of dose indicator with initial dose count of 120.

FIGS. 21, 22 and 23 shows the operation of a units wheel.

FIGS. 24A-25B shows the operation of units wheel along with the tens wheel.

FIGS. 26A-28B shows the operating cycle of dose feedback mechanism with initial dose count of 120 doses leading up to the 119^{th} dose

FIGS. 29A-31B shows the operating cycle of dose feedback mechanism when dose count reaches to 100 doses showing illustration of operation of hundreds wheel.

### DETAILED DESCRIPTION

The present disclosure provides for mechanisms and assemblies for providing dose related feedback to the user of the medicament dispenser and the physical interfaces and attributes associated with providing such feedback.

A medicament dispensers, as disclosed herein, have a variety of structural configurations and can be used for dispensing liquids, powders, tablets, capsules, pellets or pucks, or mixtures thereof for nasal, pulmonary or oral administration. In particular, the medicament dispensers can be used to dispense a liquid comprising one or more active pharmaceutical ingredient(s) (API) and optionally, one or more pharmaceutically acceptable carrier(s) and/or excipient(s). The dispenser may dispense a propellant based pressurized inhalation aerosol comprising one or more active pharmaceutical ingredient(s) (API) and optionally, one or more propellant, cosolvent, solubilizer, emulsifier, surfactant, salt, acid, and micronized or non-micronized pharmaceutically acceptable carrier(s) and/or excipient(s), wherein the dispensing process includes a press-and-breathe type, breath-actuated, or other multi-step operation medicament dispensers. Further, the present disclosure relates to dispensing powdered medicament wherein the dispensing process includes a press-and-breathe type or breath-actuated or other multi-step operation medicament dispensers.

One or more active pharmaceutical ingredient(s) (APIs) that can be used can be selected from analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone e.g. as the furoate ester), ciclesonide, triamcinolone (e.g. as the acetonide) or 6a,9a-difluoro-1 ip-hydroxy-16a-methyl-3-oxo-17a.-propionyloxy-androsta-1,4-diene-17P-carbothioic acid S-(2-oxo-tetrahydro-furan-3-yl) ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl] sulfonyl]ethyl]amino]ethyl-2(3H)-benzothiazolone; adenosine 2a agonists, e.g. 2R,3R,4S,5R)-2-[6-Amino-2-(1 S-hydroxymethyl-2-phenyl-ethylamino)-purin-9-yl]-5-(2-ethyl-2H-tetrazol-5-yl)-tetrahydro-furan-3,4-diol (e.g. as maleate); a4 integrin inhibitors e.g. (2S)-3-[4-({[4-(aminocarbonyl)-1-piperidinyl] carbonyl}oxy)phenyl]-2-[((2S-)-4-methyl-2-{[2-(2 methylphenoxy)acetyl]amino}pentanoyl)amino]propanoic acid (e.g. as free acid or potassium salt), diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimize the activity and/or stability of the medicament

The term “dose feedback mechanism” includes the mechanism by which the dose related (e.g., number remaining, number consumed, and/or life-cycle of the product) visual feedback (e.g., read-out) is provided to the user.

In some embodiments, the dose feedback mechanism includes the actuator, the dose indicator assembly, the balancer, and the window.

An actuator houses a dose indicator assembly, the canister, bottle or medicament carrier (such as reel of blisters) containing the formulation, the balancer and the window. The actuator may contains of one or more linear rack guides that allow for the linear rack of the dose indicator assembly to move in a rectilinear motion during the operation/actuation of the medicament dispenser. The actuator may have pins to locate the one or more wheels of the dose counter assembly and the balancer.

The rectilinear motion means a straight-line motion. A body is said to experience rectilinear motion if any two particles of the body travel the same distance along two parallel straight lines with uniform velocity. The rectilinear motion may constitute reciprocal motion.

In some embodiments, the dose feedback mechanism includes the dose indicator assembly having a disc mount, one or more linear racks having teeth, one or more pinions and/or a dose indicating means for providing a dose related visual feedback to the user of the medicament dispenser.

The disc mount holds and locates all the components of the dose indicator assembly except linear rack and spring. The disc mount comprises one or more of the units wheel seat, the pinion seat, the tens wheel drive gear stud, the hundreds wheel drive gear stud, locking pin mount. The disc mount enables the positioning of the dose indicator assembly within the body of the actuator. The units wheel seat and the pinion seat provide for a frictionless rotation of the units wheel and the central pinion respectively. The tens wheel drive gear stud and the hundreds wheel drive gear stud locate the tens wheel drive gear and the hundreds wheel drive gear respectively. The locking pin mount that engages with the locking pin hundreds wheel to prevent the motion of the hundreds wheel and thereby preventing any further motion of the units wheel and tens wheel.

A “linear rack” includes a linear gear bar and has teeth for engagement. A linear rack has an arrangement to attach a spring to assist the return movement of the linear rack after actuation/operation.

A “pinion” includes a circular gear and may have teeth and/or radially placed driving studs. The pinion may have ratchets. The ratchets may be present on the circumference (e.g., circumferentially placed) or along the radius (e.g., radially placed) of the pinion.

In general a linear rack and pinion are a pair of gears which convert rectilinear motion into rotational motion and vice versa.

The term “ratchet” includes a type of gear or arrangements of teeth in such way that it allows continuous linear or rotary motion in only one direction while preventing motion in the opposite direction.

A dose indicating means includes dose indicator wheel(s) or disk(s). As used herein the terms wheels or disks can be used interchangeably.

A dose indicating means which includes dose indicator wheels including one to three dose indicator wheels. The dose indicator wheels each representing the units digits, the tens digits and/or the hundreds digits. Each dose indicator wheel is imprinted, etched, pasted or embossed to provide visual information in the form of digits. The dose indicator wheels are arranged in a concentric, planar and co-axial (e.g. same axis of rotation) or non-concentric and non-planar orientation or concentric, co-planar and co-axial. The wheels may be in an overlapping or non-overlapping configuration for providing a count ranging from numbers ‘0’ to ‘999’ which are displayed synchronously, collectively and simultaneously with single or multiple components of the dose indicating means wherein the numbers ‘0’ to ‘999’ are displayed in a vertical, horizontal orientation. The dose indicator wheels may have one or more pins, studs, gears and/or ratchets.

The primary packaging in case of MDI refers to a bottle, canister or actuator, and its components such as mouth piece cover.

The balancer transfer distributes and equalizes the force of primary packaging on the linear racks. The balancer resides on top of the linear rack of the dose indicator assembly. The rectilinear or rotational motion of the primary packaging material (e.g. canister, bottle or cover) is transferred to the dose indicator assembly via the balancer. The balancer can have different shapes such as disc type, half-moon and/or bar type. The balancer may have one or more slot(s) for linear racks. The balancer may have one or more slot(s) for actuator pins. During the course of actuation/operation the actuator pins for balancer moves within the balancer slots preventing the rocking and/or shaking of the balancer.

In some embodiments, the dose feedback mechanism include the dose indicator assembly includes: a disc mount, one or more linear racks having teeth, one or more pinions, and/or a dose indicating means which include dose indicator wheels comprising one or more dose indicator wheels for providing a dose-related visual feedback to the user of the medicament dispenser.

In some embodiments, the dose feedback mechanism include of the dose indicator assembly includes: a disc mount, one or more linear racks having teeth, central pinion, and/or a dose indicating means which include dose indicator wheels comprising of one or more dose indicator wheels for providing a dose related visual feedback to the user of the medicament dispenser. The central pinion and dose indicator wheels are concentric.

In some embodiments, the dose feedback mechanism include the dose indicator assembly includes: a disc mount includes the units wheel seat, the central pinion seat, the tens wheel drive gear stud, the hundreds wheel drive gear stud and/or locking pin mount, one or more linear racks having teeth, one or more pinions, and/or a dose indicating means which includes dose indicator wheels comprising of one or more dose indicator wheels each representing the units digits, the tens digits and/or the hundreds digits for providing a dose related visual feedback to the user of the medicament dispenser.

In some embodiments, the dose feedback mechanism include the dose indicator assembly includes: a disc mount includes the units wheel seat; the central pinion seat; the tens wheel drive gear stud; the hundreds wheel drive gear stud and/or locking pin mount, one or more linear racks having teeth, central pinion, and/or a dose indicating means which include dose indicator wheels comprising of one or more dose indicator wheels each representing the units digits, the tens digits and/or the hundreds digits for providing a dose related visual feedback to the user of the medicament dispenser. The central pinion and dose indicator wheels are concentric.

In some embodiments, the dose feedback mechanism include of the dose indicator assembly includes: a disc mount including the units wheel seat; the pinion seat; the tens wheel drive gear stud; the hundreds wheel drive gear stud and/or locking pin mount, one or more linear racks having teeth, central pinion having ratchets, and/or a dose indicating means which include dose indicator wheels comprising of one or more dose indicator wheels each representing the units digits, the tens digits and/or the hundreds digits for providing a dose related visual feedback to the user of the medicament dispenser. The central pinion and dose indicator wheels are concentric. The linear rack prevents an inadvertent miss in the transfer and distribution of motion of the primary packaging within the dose feedback mechanism and prevent a false higher remaining dose count within the medicament dispenser.

In some embodiments, the dose feedback mechanism include the dose indicator assembly includes: a disc mount includes the units wheel seat, the pinion seat; the tens wheel drive gear stud, the hundreds wheel drive gear stud; and/or locking pin mount, a linear rack having teeth, a central pinion having ratchets, and/or a dose indicating means which includes dose indicator wheels comprising of one or more dose indicator wheels each representing the units digits, the tens digits and/or the hundreds digits for providing a dose related visual feedback to the user of the medicament dispenser. The central pinion and dose indicator wheels are coaxial. The linear rack prevents an inadvertent miss in the transfer and distribution of motion of the primary packaging within the dose feedback mechanism and prevent a false higher remaining dose count within the medicament dispenser.

In some embodiments when the primary packaging e.g. canister or bottle are depressed to actuate/operate the medicament dispenser the motion is transferred to the linear rack which moves it in downward direction, simultaneously and synchronously. The motion of the linear rack is transferred to the central pinion allowing it to rotate. The central pinion has circumferential ratchets that drive the units wheel. The rotation of the units wheel leads to rotation the tens wheel via the tens wheel drive gear which further rotates the hundreds wheel via hundreds wheel drive gear. The spring assists in the return movement of the linear rack after actuation/operation. The circumferential ratchets of the central pinion prevent the reverse rotation of the wheels during the return movement of the linear rack. The mechanism of actuation leads to change in the number of doses (increment or decrement) and provides dose related feedback (number remaining, number consumed and/or life-cycle of the product) to the user of the medicament dispenser.

When the primary packaging (e.g. cover of dry powder inhaler) is opened to actuate/operate the medicament dispenser the motion is transferred to the linear rack which moves it in downward direction, simultaneously and synchronously. The motion of the linear rack is transferred to the central pinion allowing it to rotate. The central pinion has circumferential ratchets that drive the units wheel. The rotation of the units wheel leads to rotation the tens wheel via the tens wheel drive gear which further rotates the hundreds wheel via hundreds wheel drive gear. The spring assist in the return movement of the linear rack after actuation/operation. The circumferential ratchets of the central pinion prevent the reverse rotation of the wheels during the return movement of the linear rack. The mechanism of actuation leads to a change in the number of doses (increment or decrement) and provides dose related feedback (e.g. number remaining, number consumed and/or life-cycle of the product) to the user of the medicament dispenser.

In some embodiments, the dose feedback mechanism include the dose indicator assembly includes: the a disc mount, one or more linear racks having teeth, a central pinion configured to convert rectilinear motion of the one or more linear racks to rotary motion, and a dose indicating means for providing a dose related visual feedback to the user of the medicament dispenser, the dose indicating means comprising a plurality of dose indicator wheels, the plurality of dose indicator wheels arranged in a concentric and co-planar configuration.

The presence of linear rack prevents an inadvertent miss in the transfer and distribution of motion of the primary packaging within the dose feedback mechanism and prevents a false higher remaining dose count within the medicament dispenser.

The presence of one or more linear rack allows for an efficient and robust transfer and distribution of motion from the actuation/operation of the medicament dispenser to and within the dose indicator mechanism and allow for relative serial reduction in the unit count of doses remaining in the medicament dispenser.

In some embodiment, the dose feedback mechanism includes: the actuator, the dose indicator assembly include disc mount; one or more linear racks having teeth; central pinion and/or a dose indicating means which include dose indicator wheels comprising of one or more dose indicator wheels for providing a dose related visual feedback to the user of the medicament dispenser, the balancer, and/or the window for displaying the change in the number of doses (increment or decrement) and provides dose related feedback (e.g. number remaining, number consumed and/or life-cycle of the product) to the user of the medicament dispenser.

In some embodiment, the dose feedback mechanism includes: the actuator having units wheel locator pin, the dose indicator assembly include a mount which further includes the units wheel seat; the pinion seat; the tens wheel drive gear stud; the hundreds wheel drive gear stud and/or locking pin mount, a linear rack having teeth, a central pinion having ratchets, and/or a dose indicating means which further includes dose indicator wheels comprising of one or more dose indicator wheels each representing the units digits, the tens digits and/or the hundreds digits for providing a dose related visual feedback to the user of the medicament dispenser, the balancer, and/or the window for displaying the change in the number of doses (increment or decrement) and provides dose related feedback (e.g. number remaining, number consumed and/or life-cycle of the product) to the user of the medicament dispenser.

It is intended that the scope of the present disclosure should not be limited by any particular embodiment described herein. While various embodiments have been described above, it should be noted that they have been presented by way of example only, and not limitation. Numerous changes to the disclosed embodiments can be made in accordance with the disclosure herein without departing from the spirit or scope of the invention.

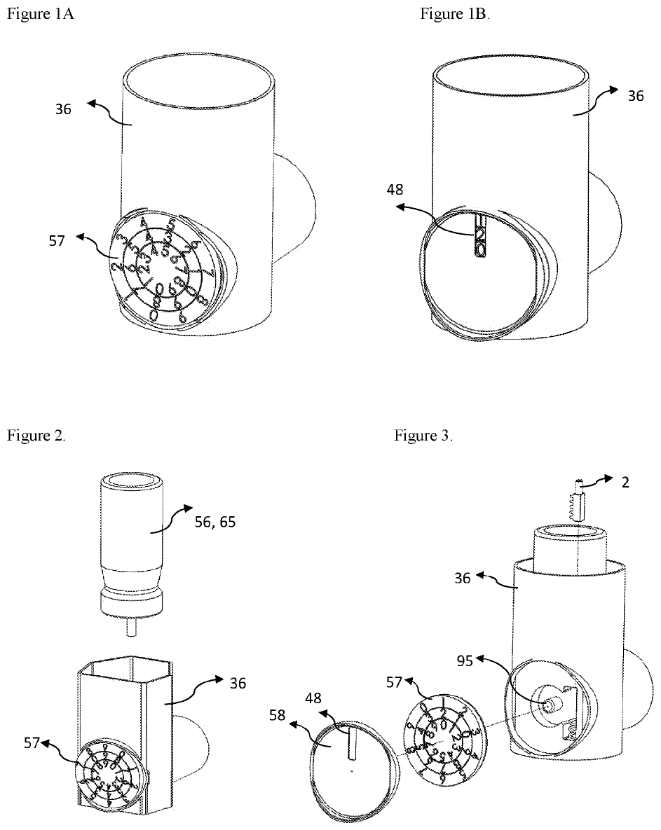

FIGS. 1A and 1B show actuator 36 for a metered dose inhaler showing a window 48 and dose indicator assembly 57. A canister 56 or bottle 65 can be inserted in to the actuator 36.

FIG. 2 shows another type of actuator 36 for a metered dose inhaler showing a dose indicator assembly 57. A canister 56 or bottle 65 can be inserted in to an actuator.

FIG. 3 shows an exploded view of the medicament dispenser showing actuator 36, dose indicator assembly 57, units wheel locator pin 95, dose indicator assembly cover 58 and window 48. The dose indicator assembly 57 is nestled within the dose indicator assembly cover 58. The dose indicator cover 58 can be molded, bonded or attached to the actuator 36 body through mechanical means. The dose indicator cover 58 bears the window 48.

In some embodiments, the dose indicator assembly 57 can also be adapted as a stand-alone cartridge/cassette for insertion into the molded body of the actuator 36 for the ease of manufacturing operation.

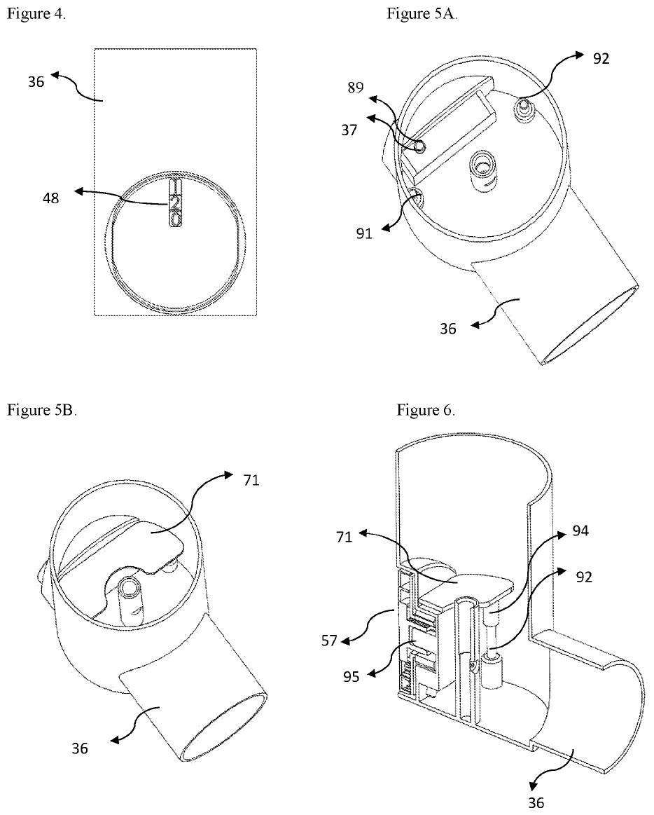

FIG. 4 shows the view of window 48 displaying the count of the dose indicator assembly 57 through the window 48.

FIGS. 5A and 5B show top isometric views of an actuator showing linear rack guides 37 for linear rack 2 having linear rack pin 89, balancer locator pin left 91, balancer locator pin right 92 and bar type balancer 71. The balancer 71 locates over the balancer locator pin left 91 and balancer locator pin right 92 on the actuator 36 body (see FIG. 6). The balancer locator pin left 91 and the balancer locator pin right 92 guide the balancer 71 during the actuation. During the course of actuation/operation, the balancer locator pin left 91 and balancer locator pin right 92 move within the balancer guide left 93 and the balancer guide right 94, respectively. The rectilinear motion of the canister 56 or bottle 65 within the body of the actuator 36 is transferred to the dose indicator assembly 57 via the balancer 71. The operation/actuation of the medicament dispenser includes rectilinear motion of the canister 56 or bottle 65 within the body of the actuator 36 transferred to the linear rack 2 on the dose indicator assembly 57 via the balancer 71.

FIG. 6 shows a sectional view showing the actuator 36, bar type balancer 71, dose indicator assembly 57, balancer locator pin right 92, balancer guide right 94 and units wheel locator pin 95.

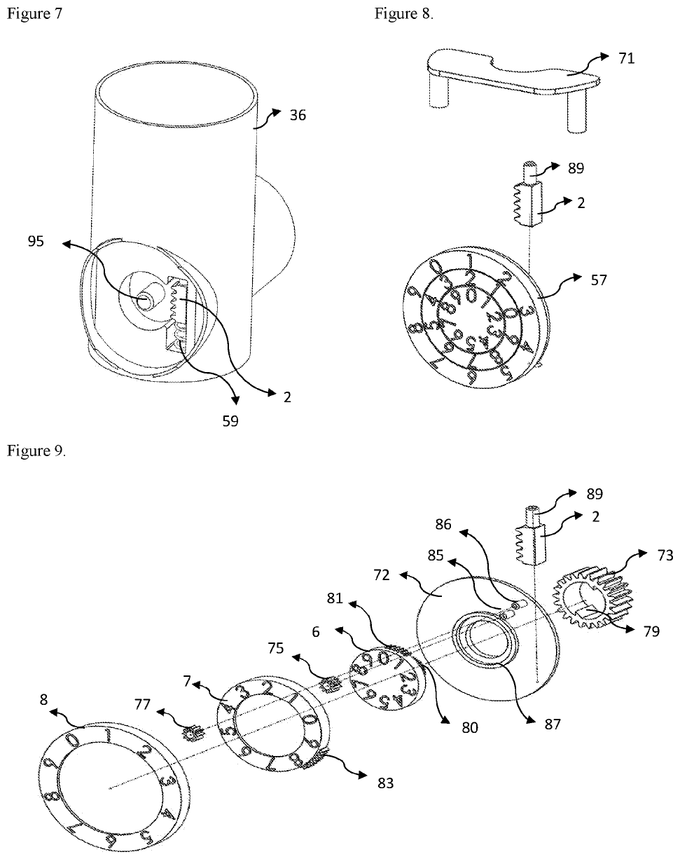

FIG. 7 shows actuator 36, linear rack 2, spring 59 and units wheel locator pin 95. The figure shows the assembly of linear rack 2 and spring 59 inside the actuator. The spring 59 assists in the return movement of the linear rack 2 after the actuation/operation.

FIG. 8 shows an arrangement of dose indicator assembly 57 and bar type balancer 71. The pin 89 of liner rack is pressed down by the balancer during the actuation/operation.

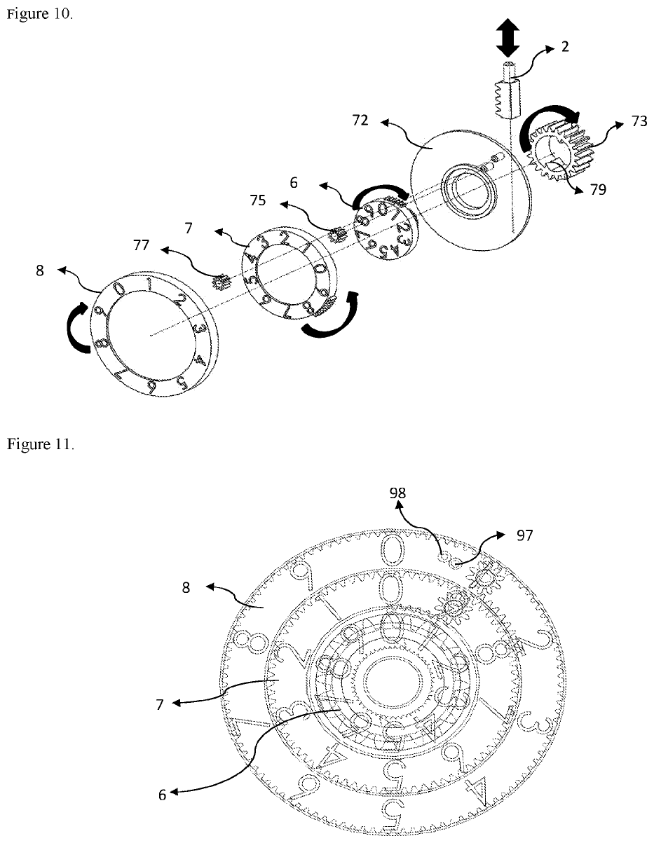

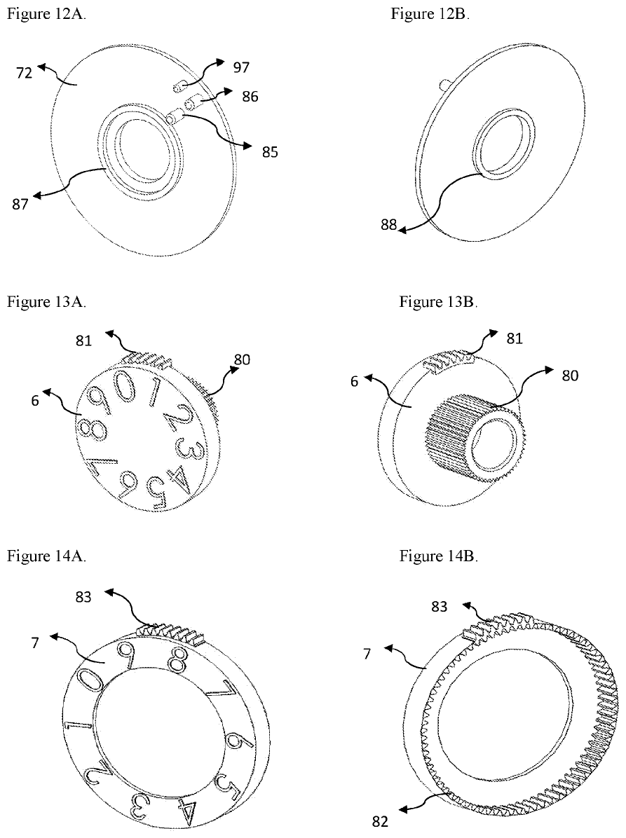

FIGS. 9 and 10 show an exploded view of an embodiment of the dose indicator assembly 57. The dose indicator assembly 57 includes the linear rack 2, spring 59 (not shown), disc mount 72, central pinion 73 have ratchets 79 on inner circumference, units wheel 6, tens wheel drive gear 75, tens wheel 7, hundreds wheel drive gear 77, hundreds wheel 8. The units wheel 6 has units wheel drive gear 80 and incrementing gears units wheel 81. The tens wheel 7 has tens wheel inner gears 82 (see FIG. 14B) and incrementing gears tens wheel 83. The hundreds wheel 8 has hundreds wheel inner gears 84 and locking pin hundreds wheel 98 (see FIG. 15B). The disc mount 72 holds and locates all the components of the dose indicator assembly except linear rack 2 and spring 59. The disc mount 72 enables the positioning of the dose indicator assembly within the body of the actuator 36. The disc mount 72 includes a units wheel seat 87 and the pinion seat 88 (see FIG. 12B) that provide for a frictionless rotation of the units wheel 6 and the central pinion 73, respectively. The disc mount 72 includes the tens wheel drive gear stud 85 (see FIG. 12A) and the hundreds wheel drive gear stud 86 (see FIG. 12A) that locate the tens wheel drive gear 75 and the hundreds wheel drive gear 77, respectively. The disc mount 72 may include the locking pin mount 97 (see FIG. 12A) that engages with the locking pin hundreds wheel 98 to prevent the motion of the hundreds wheel 8 and thereby preventing any further motion of the hundreds wheel 8 (see FIG. 11). This dose indicator assembly 57 allows for optional dose lock-out and operation lock-out feature at the stage of exhaustion of the doses in the medicament dispenser. The linear rack has linear rack pin 89 (see FIG. 18) which guides the rack 2 through the actuator linear rack guide 37 on the actuator 36 (see FIG. 5A). This dose indicator set-up allows for a three digit (units) read-out and feedback on dose numbers in a medicament dispenser ranging from ‘0’ to up to ‘999’.

The spring 59 (not shown) compresses when the canister 56 or bottle 65 is depressed either by the press-and breathe operation of the user or the activation of the breath triggered actuation mechanism, both resulting in downward rectilinear motion of the canister 56 or bottle 65 and remains in the state of compression as long as the canister 56 or bottle 65 is depressed. When the canister 56 or bottle 65 is not depressed and returns back to its original state, the spring 59 (not shown) uncompresses and moves the linear rack 2 vertically to its original state. The central pinions 73 rotate both clockwise and counter-clockwise due to the to-and-fro motion of the linear rack 2. When the linear rack 2 moves in a downward motion, simultaneously and synchronously, the central pinion 73 moves in a clock-wise motion. When the linear rack 2 moves in an upward motion, simultaneously and synchronously, the central pinion 73 moves in a counter-clockwise motion. The units wheel 6 is driven by the ratchets 79 on the central pinion 73. The ratchets 79 on the central pinion 73 drive the units wheel drive gear 80 unidirectionally preventing reverse rotation thereby incrementing the units wheel 6. The incrementing gears units wheel 81 comes in contact with the tens wheel drive gear 75 after every 10 actuations/operations or after one complete rotation of the units wheel 6. The tens wheel drive gear 75 is always in contact with the tens wheel inner gears 82 (see FIG. 14B) of the tens wheel 7. The tens wheel 7 is driven by the incrementing gears units wheel 81 on the units wheel 6 via the tens wheel drive gear 75. The hundreds wheel 8 is driven by the incrementing gears tens wheel 83 on the tens wheel 7 via the hundreds wheel drive gear 77. The hundreds wheel drive gear 77 is always in contact with the hundreds wheel inner gears 84 (see FIG. 15B) of the hundreds wheel 8. The hundreds wheel 8 increments when the incrementing gears tens wheel 83 comes in contact with the hundreds wheel drive gear 77 after every complete rotation of the tens wheel 7 or every 100 actuations/operations.

FIG. 11 shows an illustration of dose lockout feature. In some embodiments, the dose indicator mechanism is also characterized by an optional dose lock-out and operation lock-out feature at the stage of exhaustion of the doses in the medicament dispenser. When the dose indicator reads ‘000’, the locking pin 98 on the hundreds wheel 8 comes in contact with the locking pin mount 97 on the disc mount 72. At this stage, the tens wheel 7 cannot increment past ‘0’ since the incrementing gears tens wheel 83 on the tens wheel 7 are not able to rotate the hundreds wheel drive gear 77 which is always in contact with the hundreds wheel inner gears 84 of the hundreds wheel 8. Similarly, the units wheel 6 cannot increment past ‘0’ since the incrementing gears units wheel 81 on the units wheel 6 are not able to rotate the tens wheel drive gear 75 which is always in contact with the tens wheel inner gears 82 of the tens wheel 7.

FIGS. 12A and 12B show different isometric views of disc mount 72 showing the tens wheel drive gear stud 85 and the hundreds wheel drive gear stud 86, units wheel seat 87, the pinion seat 88 and/or locking pin mount 97.

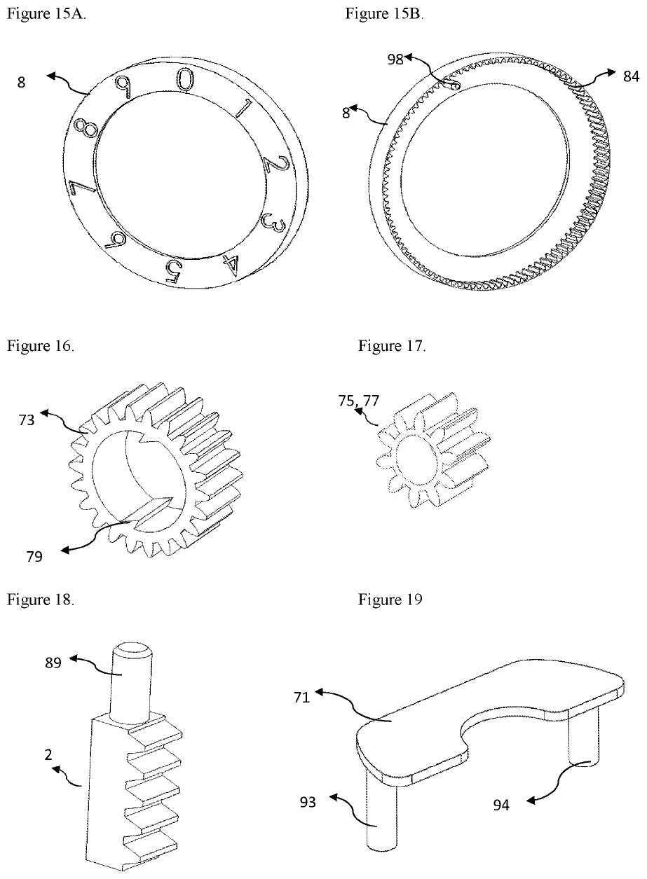

FIGS. 13A and 13B show different isometric views of units wheel 6. The units wheel 6 showing units wheel drive gear 80 and incrementing gears units wheel 81.

In some embodiments, the units digits are on a front face of the units wheel opposite the units wheel drive gear 80, not the peripheral edge of the units wheel.

FIGS. 14A and 14B show different isometric of tens wheel 7. The tens wheel 7 showing tens wheel inner gears 82 and incrementing gears tens wheel 83.

In some embodiments the tens digits are on a front face of the tens wheel, not the peripheral edge of the tens wheel.

FIGS. 15A and 15B show different isometric view of hundreds wheel 8. The hundreds wheel 8 have hundreds wheel inner gears 84 and locking pin hundreds wheel 98. The hundreds digits are on a front face of the hundreds wheel.

FIG. 16 shows an isometric view of central pinion showing ratchets 79 placed on inner circumference of the central pinion.

FIG. 17 shows an isometric view of tens/hundreds wheel drive gear 75 and 77.

FIG. 18 shows isometric view of linear rack 2 showing linear rack pin 89.

FIG. 19 shows an isometric views of bar type balancer 71 showing balancer guide left 93 and the balancer guide right 94. During the course of actuation/operation the balancer locator pin left 91 and balancer locator pin right 92 (see FIG. 5A) on balancer move within the balancer guide left 93 and the balancer guide right 94 and guide the movement of the balancer and prevent the rocking and/or shaking of the balancer in both the idle state and during the operation.

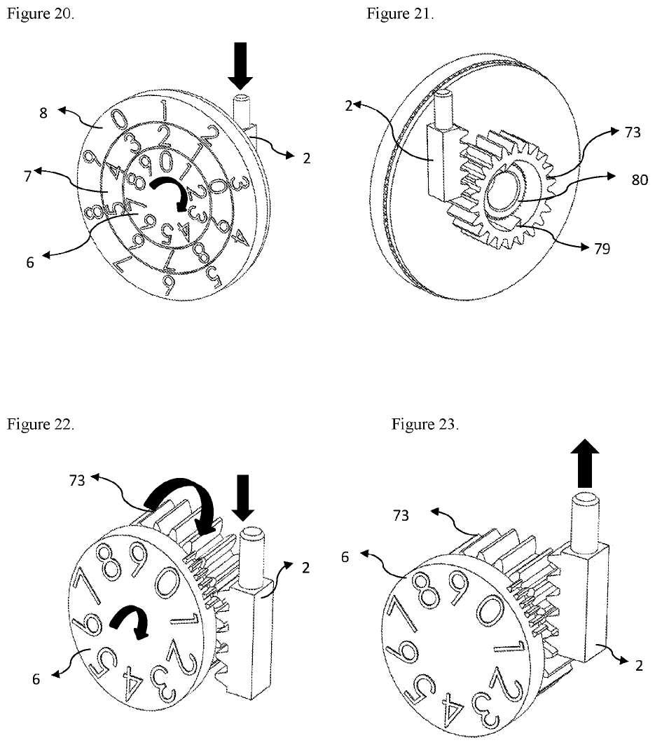

FIG. 20 shows the operating cycle of dose indicator 57 with initial dose count of 120.

FIGS. 21, 22 and 23 show the operation of units wheel 6. The tens wheel 7 and the hundreds wheel 8 are not shown for clarity in the figures. During each actuation/operation, the downward rectilinear motion of the canister 56 or bottle 65 (not shown) leads to recti-linear motion of the linear rack 2 which leads to clock-wise movement of the central pinion 73. The central pinion 73 has ratchets 79 that drives the units wheel drive gear 80 uni-directionally (clockwise) (see FIGS. 21 and 22). At the end of the dosing cycle when the canister 56 or bottle 65 (not shown) is no longer depressed the linear rack 2 moves in the upward direction to return back to its rest state due to spring 59 (not shown). At this stage there is no movement of the units wheel 6 because of the ratchet mechanism of the central pinion 73 (see FIG. 23). The units wheel rotates after each actuations/operations.

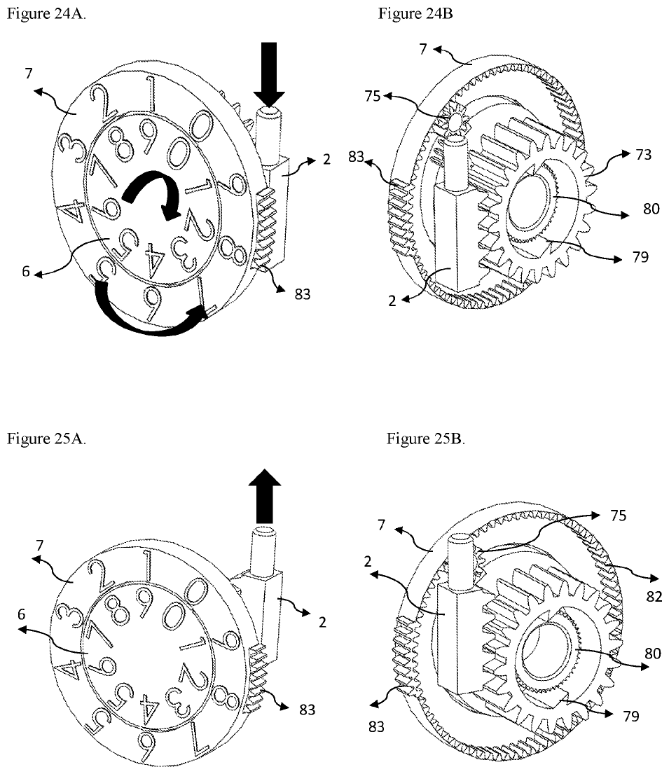

FIGS. 24A, 24B, 25A and 25B show the operation of units wheel along with the tens wheel of dose indicator 57 with initial dose count of 120. FIGS. 24B and 25B shows an isometric view of FIGS. 24A and 25A from another side to show the position and interaction between the various components respectively. The hundreds wheel 8 is not shown for clarity in the figures. During each actuation/operation, the downward rectilinear motion of the canister 56 or bottle 65 (not shown) leads to recti-linear motion of the linear rack 2 which leads to clock-wise movement of the central pinion 73. The central pinion 73 has ratchets 79 that drives the units wheel drive gear 80 uni-directionally (clockwise). The incrementing gears units wheel 81 on the units wheel 6 drives the tens wheel 7 via the tens wheel drive gear 75. The tens wheel inner gears 82 are in contact with the tens wheel drive gear 75. The tens wheel 7 increments when the tens wheel drive gear 75 comes in contact with the incrementing gears units wheel 81 on the units wheel 7. At the end of the dosing cycle when the canister 56 or bottle 65 (not shown) are no longer depressed the linear rack 2 moves in the upward direction to return back to its rest state due to spring 59 (not shown). At this stage there is no movement of the units wheel 6 because of the ratchet mechanism of the central pinion 73 and thereby no further movement of the tens wheel 7 (see FIG. 25). The units wheel rotates after each actuations/operations. The tens wheel 7 rotates after every 10 actuations/operations or after one complete rotation of the units wheel 6.

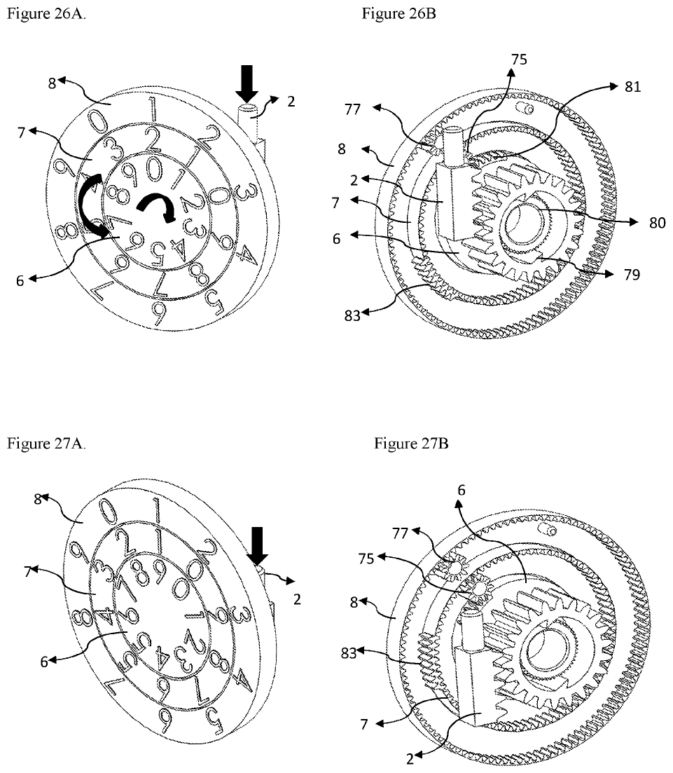

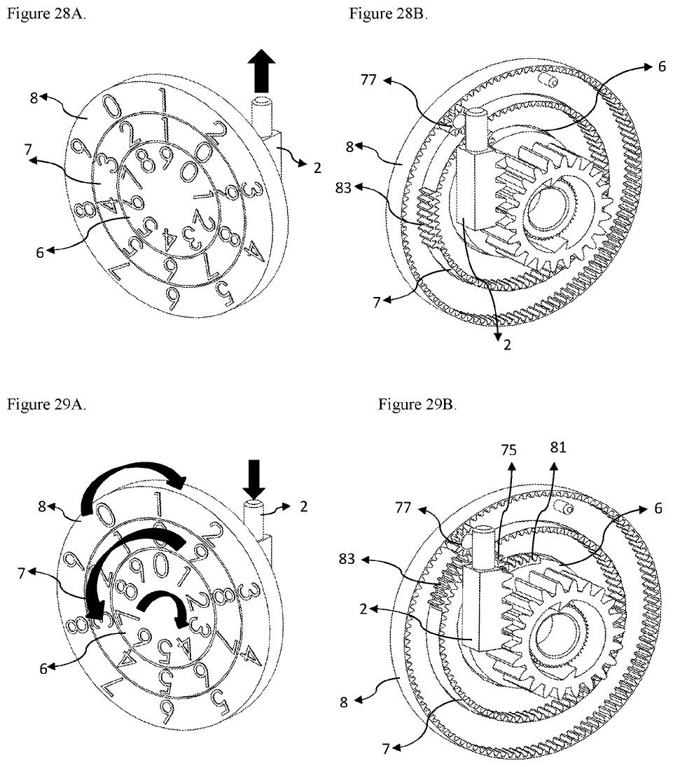

FIGS. 26A, 26B, 27A, 27B, 28A and 28B show the operating cycle of dose feedback mechanism with initial dose count of 120 doses leading up to the 119^{th} dose. FIGS. 26B, 27B and 28B show an isometric view of FIGS. 26A, 27A and 28A from another side to show the position and interaction between the various components respectively. At this stage, the dose counter 57 reads ‘120’ thereby displaying ‘0’ on units wheel 6, ‘2’ on tens wheel 7 and ‘1’ on hundreds wheel 8. When the canister 56 or bottle 65 is depressed to actuate/operate the medicament dispenser for the first time (once), the motion is transferred to the linear rack 2. At this stage, the linear rack 2 move in a downward motion, which leads to clock-wise movement of the central pinion 73. The central pinion 73 has ratchets 79 that drive the units wheel drive gear 80 uni-directionally (clockwise) (see FIGS. 26A and 26B). At this stage the units wheel 6 move by decrementing from ‘0’ to ‘9’ (see FIGS. 27A and 27B). At the same time the incrementing gears units wheel 81 on the units wheel 6 drives the tens wheel 7 via the tens wheel drive gear 75 which is always in contact the tens wheel inner gears 82; thereby rotating and decrementing the tens wheel 7 from ‘2’ to ‘1’ (see FIGS. 27A and 27B). During this rotation of the tens wheel 7 from ‘2’ to ‘1’, the incrementing gears hundreds wheel 83 on the tens wheel 7 does not come in contact the hundreds wheel 8. As a result, the hundreds wheel 8 does not rotate and continues to displays ‘1’. At the end of this actuation/operation, the dose counter will display ‘119’ (see FIG. 27A). Once the canister 56 or bottle 65 is no longer depressed, the linear rack 2 moves in an upward motion due to the spring 59 (not shown) and achieves the original position. At this stage there is no further movement of the units wheel 6 or dummy wheel 9 or tens wheel 7 or hundreds wheel 8 (see FIGS. 28A and 28B). The dose indicator will continue to move during each successive actuation/operation by decrementing the units wheel from ‘9’ to ‘0’. The units wheel 6 and the incrementing gears units wheel 81 on the units wheel 6 does not come in contact with tens wheel 7 for the next ten actuations/operations. As a result, the tens wheel 7 will continue to display ‘1’ and also the hundreds wheel 8 will continue to display ‘1’ till one complete rotation of tens wheel.

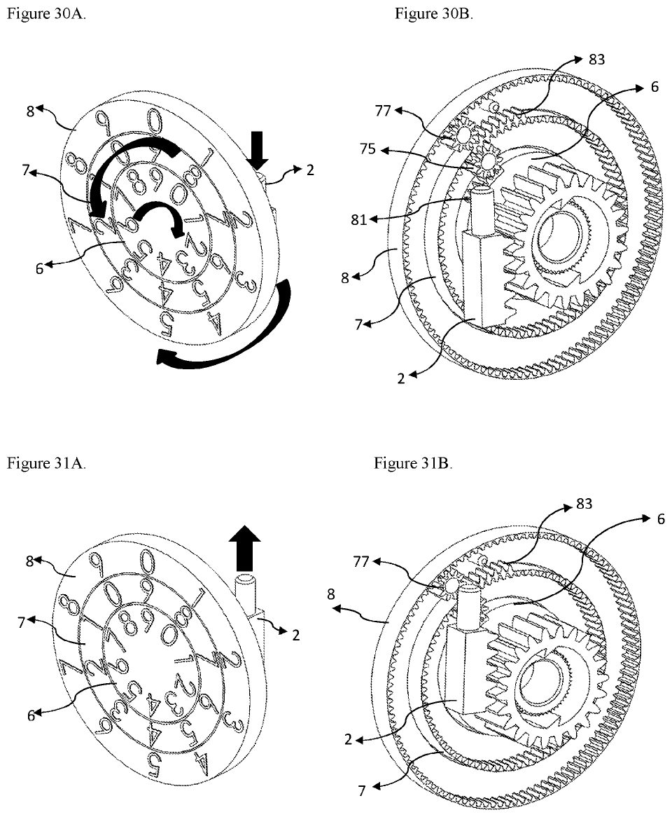

FIGS. 29A, 29B, 30A, 30B, 31A and 31B show the operating cycle of dose feedback mechanism when dose count reaches to 100 doses showing illustration of operation of hundreds wheel. FIGS. 29B, 30B and 31B shows an isometric view of FIGS. 29A, 30A and 31A from another side to show the position and interaction between the various components respectively. When the dose indicator reads ‘100’ and the canister 56 or bottle 65 is depressed to actuate/operate, at this stage, the units wheel decrements from ‘0’ to ‘9’ (FIGS. 29A, 30A). When the units wheel 6 decrements from ‘0’ to ‘9’, the incrementing gears units wheel 81 on the units wheel 6 drives the tens wheel 7 via the tens wheel drive gear 75 which is always in contact the tens wheel inner gears 82; thereby rotating and decrementing the tens wheel 7 from ‘0’ to ‘9’. When the tens wheel 7 decrements from ‘0’ to ‘9’, the incrementing gears hundreds wheel 83 on the tens wheel 7 come in contact the hundreds wheel 8 via the hundreds wheel inner gears 84 of the hundreds 8 and hundreds wheel drive gear 77 thereby rotating and decrementing the hundreds wheel 8 from ‘1’ to ‘0’. At the end of this actuation/operation, the dose counter displays ‘099’. Once the canister 56 or bottle 65 is no longer depressed, the linear rack 2 moves in an upward motion due to the spring 59 (not shown) and achieve the original position. At this stage there is no further movement of the units wheel 6 or dummy wheel 9 or tens wheel 7 or hundreds wheel 8 (see FIG. 31A). The counter still displays ‘099’. The units wheel 6 increments during every actuation/operation. The tens wheel 7 increments after each 10 actuation/operation e.g., after one complete rotation of the units wheel 7. Similarly, the hundreds wheel 8 increments after every 100 actuation/operation i.e. after one complete rotation of the tens wheel 7 or ten complete rotations of the units wheel 6.

1. A dose indicator assembly comprising:
a disc mount;
one or more linear racks having teeth;
a central pinion configured to convert rectilinear motion of the one or more linear racks to rotary motion; and
a dose indicating means comprising plurality of dose indicator wheels arranged in a concentric and co-planar configuration.

2. The dose indicator assembly according to claim 1, wherein the dose indicating means provides a count ranging from numbers ‘0’ to ‘999’.

3. The dose indicator assembly according to claim 2 wherein the numbers ‘0’ to ‘999’ are displayed in a vertical, horizontal orientation.

4. The dose indicator assembly according to claim 1, wherein one or more dose indicator wheels comprises a units wheel, a tens wheel and a hundreds wheel.

5. The dose indicator assembly according to claim 1, wherein the dose indicator assembly further comprises: a units wheel seat, a pinion seat, a tens wheel drive gear stud, a hundreds wheel drive gear stud and a locking pin mount.

6. The dose indicator assembly according to claim 4, wherein the units wheel is rotated by the central pinion.

7. The dose indicator assembly according to claim 6, wherein the tens wheel is rotated by the units wheel via the tens wheel drive gear.

8. The dose indicator assembly according to claim 7, wherein the tens wheel rotates the hundreds wheel via the hundreds wheel drive gear.

9. The dose indicator assembly according to claim 8, wherein the unit wheel rotates each time the central pinion rotates and the tens wheel rotate once on every tenth rotation of units wheel.

10. The dose indicator assembly according to claim 9, wherein the hundreds wheel rotates once on every tenth rotation of the tens wheel.

11. The dose indicator assembly according to claim 1, further comprises operation lock-out mechanism.
